(19) United States
(12) Patent Application Publication
(10) Pub. No.: US 2022/0347075 A1
(43) Pub. Date: Nov. 3, 2022

(54) PEPTIDE BASED COSMETIC OR DERMATOLOGICAL TREATMENT OF THE SKIN AND ITS APPENDAGES

(71) Applicant: Sederma, Le Perray en Yvelines (FR)

(72) Inventors: Philippe Mondon, Montrouge (FR); Caroline Ringenbach, Rambouillet (FR); Thibault Marchand, Castres (FR)

(73) Assignee: Sederma, Le Perray en Yvelines (FR)

(21) Appl. No.: 17/765,707

(22) PCT Filed: Oct. 6, 2020
(86) PCT No.: PCT/EP2020/077974

§ 371 Date: Mar. 31, 2022

(30) Foreign Application Priority Data
Oct. 7, 2019 (FR) FR1911097

(51) Int. Cl. A61K 8/64; A61K 8/39; A61K 8/81; A61Q 19/00

(52) U.S. Cl. CPC A61K 8/64; A61K 8/39; A61K 8/8147; A61Q 19/00

(57) ABSTRACT

The treatment according to the invention provides the use of at least one peptide having the general Formula X-(Xaa)nK*TTK*X′aa-(Xaa)m-Z for a non-therapeutic cosmetic treatment of keratinous tissues of the skin and of its appendages, where X, Xaa, n, K*, X′aa, m and Z are as defined. A preferred peptide is the Pal-KTTKS.

### TECHNICAL FIELD

The present invention relates to a cosmetic or dermatological treatment based on peptide(s) of the skin and its appendages of mammals, human or animal.

More particularly, it concerns the cosmetic, dermatological, hygiene and personal care products industries.

### BACKGROUND ART

Peptides have an important signal function and coordinate many biochemical processes. As a result, they have become essential and promising active ingredients, more particularly in the cosmetics industry where new compounds capable of embellishing the skin and appendages are constantly being sought, for improving their general condition.

Most of the peptides currently on offer are peptides that act on the dermis by stimulating components of the extracellular matrix, mainly collagen and elastin. Many peptides are proposed in this area, in particular by the Applicant, such as the Pal-KTTKS (SEQ ID NO 1) sold under the Matrixyl® trademark, the Pal-GHK and Pal-GQPR mixture (SEQ ID NO 2) sold under the Matrixyl® 3000 trademark, the Pal-KMO₂K sold under the Matrixyl® synthe'6® trademark (MO₂ corresponding to a dioxygenated methionine), more recently the Pal-K(P)HG (with a proline grafted on a lysine) sold under the Matrixyl® Morphomics® trademark, the Pal-VGVAPG (SEQ ID NO 3) sold under the Dermaxyl™ or Biopeptide EL™ trademarks or the N-acetyl-Tyr-Arg-O-hexadecyl ester sold under the Idealift™ or Calmosensine™ trademarks.

However, beauty and good health of the skin also depend to a large extent on the quality and thickness of the epidermis, in particular through an optimal keratinocyte differentiation, and the capacity of the epidermis to form its outermost layer, the stratum corneum, and to renew it regularly by desquamation. Epidermis, and in particular the stratum corneum, forms actually a real skin barrier that is essential for protecting ourselves from molecules and attacks from the external environment (light radiation, pollutants, etc.). Thanks to a good skin barrier protection, the risks of micro-inflammations of the epidermis are for example limited, which can cause premature skin aging, this protection being even more necessary for sensitive skin. The risk of loosing water is also limited, helping to maintain good epidermis hydration. In addition to this physical barrier, there is also a chemical barrier formed by antimicrobial peptides which are synthesized by keratinocytes. Their role is to protect skin from pathogenic bacteria. It is important to preserve and improve this chemical barrier.

Besides this, the skin microbiota, a very complex ecosystem composed of a set of living microorganisms (bacteria, yeasts, viruses, and parasites), has several functions: role of defense, skin barrier and regulator of the immune system. It is important to protect the balance of skin microbiota by preventing, for example, that certain species by developing itselves excessively do not cause damage to the skin. This is the case, for example, with yeasts of the genus Malassezia, involved in dandruff states, as well as Propionibacterium acnes (recently renamed Cutibacterium acnes), the acne bacteria. The latter, although part of the normal micro-flora of the skin, by multiplying too quickly, will promote the proliferation and migration of keratinocytes, participate in the formation of radical species such as superoxide anion and cause a cascade of reactions that results in the production of pro-inflammatory molecules, and thus contribute to acne development.

Yeasts of Malassezia genus are also part of the normal micro-flora of the scalp. It is when they manage to multiply quickly enough that they cause dandruff states.

It is therefore important to preserve the balance of the skin microbiota.

### SUMMARY OF THE INVENTION

The aim of the present invention is to provide a peptide having an activity on keratinous tissues, in particular the epidermis and appendages of the skin such as the nails, the hair and the body hair (including the eyelashes and the eyebrows). In particular, the invention aim is to provide a peptide capable of acting on the stratum corneum, i.e. on the first layers on the surface of the skin, and to meet the aforementioned needs.

To this end, according to a first object, the present invention provides the use of at least one peptide of the following general Formula 1:

X-(Xaa)ₙK*TTK*X′aa-(Xaa)ₘ-Z

for a non-therapeutic cosmetic treatment of keratinous tissues of the skin and of its appendages, wherein in the general Formula 1:
K* is selected from a lysine (Lys, K), hydroxylysine, ornithine (Orn), diaminobutyric acid (Dab) or diaminopropionic acid (Dap) or their formylated, acetylated, trifluoroacetylated, methanesulfonylaed or succinylated derivatives, both K* being identical or different;
(Xaa)ₙ and (Xaa)ₘ corresponding independently of one another to a sequence of n or m amino acids Xaa selected independently of one another from a glycine (Gly, G), alanine (Ala, A), proline (Pro, P), valine (Val, V), leucine (Leu, L), isoleucine (Ile, I) and phenylalanine (Phe, F), with n and m being integers which can be equal or different and comprised between 0 and 5;
X′aa is selected from threonine (Thr, T) and serine (Ser), S);
At the N-terminal end X is selected from H, —CO—R¹, —SO₂—R¹ or a biotinoyle group;
At the C-terminal end Z is selected from OH, OR¹, NH₂, NHR¹ or NR¹R²; and
R¹ and R² being, independently of one another, selected from an alkyl, aryl, aralkyl, alkylaryl, alkoxy, saccharide and aryloxy group, which can be linear, branched, cyclic, polycyclic, unsaturated, hydroxylated, carbonylated, phosphorylated and/or sulfurated, said group having from 1 to 24 carbon atoms and the possibility of having in its backbone one or more 0, S and/or N heteroatoms.

The state of the epidermis and its appendages (such as the nails, hair, and body hair), via an action in particular on the keratinocytes, is preserved or improved thanks to the invention.

The peptide used according to the invention is characterized in that it contains at least the amino acid sequence K*TTK*X′aa, X′aa being T or S, a sequence which is biologically active on keratinocytes. Sequences of 1 to 5 non-polar amino acids chosen from Gly, Ala, Pro, Val, Leu, Ile and Phe, can be added on either side of the active sequence K*TTK*X′aa, preferably chosen from Gly, Ala and Phe, more preferably Gly and Ala.

Preferably, the derivative is an acetylated derivative, and preferably also the derivative results from a modification of the first K* at the N-terminus side of the peptide.

Preferably, according to the invention K* is a lysine K or an ornithine, more preferably a lysine.

More preferably according to the invention n and m are equal to 0, 1 or 2, preferably are equal to 0, the peptide having the general Formula 2: X-K*TTK*X′aa-Z (SEQ ID NO 4).

Preferably, the peptide according to the invention has the general Formula 3: X-KTTKX′aa-Z, X and Z and X′aa being as defined above.

A preferred example is X-KTTKS-Z, and even more preferably Pal-KTTKS (SEQ ID NO 1).

Another example of a preferred peptide is Pal-GKTTKS (SEQ ID NO 5).

Results of in vitro tests on keratinocyte culture are given below in the description showing for example, thanks to the peptide according to the invention, a preservation/protection and an improvement of the epidermis state through the strengthening of the skin barrier function and a harmonization of the natural process of maturation of the epidermis.

Proteomic and DNA-array results are also given below in the detailed description, confirming these effects on the epidermis and further showing an action of the at least one peptide according to the invention on appendages constituted of keratinocytes, such as hair, body hair (including eyelashes and eyebrows) and nails, as well as an anti-acne action.

Moreover, a specific and significant effect of the peptide according to the invention against Propionibacterium acnes has been shown in two tests involving cultures of this bacterium.

All these tests demonstrated a targeted action of the peptide according to the invention at several levels, in particular:
The stimulation of the production of several molecules constituting the skin barrier or playing a positive role in the differentiation of keratinocytes at the origin of the barrier;
The improvement of the renewal of the stratum corneum by desquamation;
The stimulation of alpha-crystalline production to protect the skin from UV, inflammatory and oxidative attacks;
The decrease in level of a certain number of molecules involved in skin microinflammations caused by the many small daily attacks (light radiation, pollutants, etc.);
The improvement of the hydration of the epidermis upper part by reducing the transepidermal loss of water (TEWL);
The treatment of epidermal scars, in particular their reduction, and in particular epidermal acne traces or marks;
An action against the bacteria responsible of acne (Propionibacterium acnes) and against dandruff yeasts (Malassezia);
An action on the hair and body hair, to improve the strength and growth of the hair;
An action on the nails;
An anti-inflammatory action (preventing action against inflammations).

Therefore, the treatment according to the invention is adapted:
To preserve, protect and/or improve the condition of the epidermis by strengthening the skin barrier function and harmonizing the natural process of epidermal maturation; and/or
To protect the skin and its appendages from molecules and agressions of the external environment including the defence against bacteria, inflammation, and radiations; and/or.
To improve the hydration of the upper part of the epidermis by reducing the transepidermal water loss (TEWL); and/or
To treat the epidermal scars, in particular to treat epidermal acne scars, in particular by reducing the appearance and visibility of the scars; and/or
To beautify nails, hair and body hair (including eyelashes and eyebrows); and/or
To fight against the microorganisms of acne and/or of dandruff state.

Thus, these results show that the use of the peptide according to the invention is particularly advantageous for treating oily and/or acne-prone skin via a preventive action. This skin type often corresponding in teen age years to an oily skin due to sebum excess for hormonal reasons. The prevention or limitation of Propionibacterium acnes bacteria proliferation prevents the occurrence, firstly of waste in the hair sheath in the hair follicle, where the bacteria develops anaerobically by feeding on sebum and producing waste, in particular dead cells, leading then to an obstruction of the hair follicle sheath (black dots formation), leading to inflammations and finally to acne pimples whose treatment falls within the scope of dermatology. Therefore, the reduction in the amount of the number of Propionibacterium acnes can prevent the progression towards a pro-acne state, in particular a pro-inflammatory state.

Also from a non-therapeutic cosmetic point of view, the results show that the use of the invention peptide is particularly suitable and advantageous for treating (reducing effect) an unsightly skin due to scars or traces of acne remaining after an acne crisis, via an epidermis renewal exercing a soft natural smoothing treatment.

Preferably, the peptide according to the invention is either modified in the N-terminal position or in the C-terminal position, preferably in the N-terminal position only.

According to other preferred features of the invention:
R¹ and/or R² is an alkyl chain of 1 to 24 carbon atoms, preferably a lipophilic alkyl chain of 3 to 24 carbon atoms; and/or
X is an acyl group CO—R′; preferably chosen from octanoyl (C8), decanoyl (C10), lauroyl (C12), myristoyl (C14), palmitoyl (C16), stearoyl (C18), biotinoyl, elaidoyl, oleoyl and lipoyl; more preferably chosen from a lauroyl (C12), myristoyl (C14) and palmitoyl (C16), and/or
Z is chosen from OH, OMe, OEt and NH₂, preferably OH; and/or
X is chosen from a palmitoyl (C16), myristoyl (C14) and lauroyl (C12); more preferably palmitoyl (C16) and Z is OH; and/or
X′aa is Serine.

Peptides comprising in the N or C terminal position derivatives of particular acids such as those of ascorbic, retinoic, cinnamic, oleanolic, hyaluronic, nicotinic, lipoic, gallic or pantothenic acid are also covered by the present invention.

A preferred peptide according to the invention is the Pal-KTTKS-OH (also called Pal-KTTKS, INCI name: Palmitoyl Pentapeptide-4, sold under the trademark Matrixyl®, SEQ ID NO 1), corresponding to a substitution with a palmitoyl chain N-terminal side (X=Pal) and no substitution on C-terminal side (Z=OH).

The peptide according to the invention may be optically pure or consist of the L or D isomers or a mixture thereof. L isomers which are those present in the natural state may be preferred. The peptide can optionally be in the form of a salt, for example a hydrochloride or acetate.

The present invention also covers derivatives of the peptide (with modification and/or addition of a chemical function but without change in the carbon skeleton) and the analogs (with modification and/or addition of a chemical function but with in addition a change in the carbon skeleton), complexes with other species such as a metal ion (eg copper, zinc, manganese, magnesium, and others).

For its use according to the invention, the peptide can be solubilized in a physiologically acceptable lipophilic or hydrophilic matrix, optionally with a solubilizer, depending on the envisaged dosage form.

By “physiologically acceptable medium” is meant according to the present invention, without limitation, an aqueous or hydroalcoholic solution, a water-in-oil emulsion, an oil-in-water emulsion, a microemulsion, an aqueous gel, an anhydrous gel, a serum, a dispersion of vesicles, a powder.

“Physiologically acceptable” means that the ingredients and compositions comprising the peptide according to the invention are suitable for topical or transdermal use, in contact with mucous membranes, nails, scalp, hair, hair and skin of mammals and more particularly human, compositions which can be ingested or injected into the skin, without risk of toxicity, incompatibility, instability, allergic response, and others. This “physiologically acceptable medium” forms what is conventionally called the excipient of the composition.

The peptide according to the invention can also be used in a vectorized form, by being bound, incorporated or adsorbed on/to macro-, micro-, or nano-particles such as capsules, spheres, liposomes, oleosomes, chylomicrons, sponges, in the form of micro- or nano-emulsions, or adsorbed for example on powdery organic polymers, talcs, bentonites, spores or exins and other inorganic or organic supports.

Thus, the present invention provides also according to a second object the use of at least one peptide according to the first object and as recited above for the preparation of a composition for treatment of keratinous tissues of the skin and of its appendages.

A composition comprising the peptide according to the invention can be provided in any galenic form (examples are given below in the description) and also be conveyed via a textile support made of natural or synthetic fibers, wool, or any suitable material to come into contact with the skin, or which can be used in clothing, such as day or night underwear, handkerchiefs, or fabrics, in order to exert its cosmetic or dermatological effect through this skin/fabric contact and allow continuous topical delivery.

In a particular and advantageous manner, according to the invention, the peptide can be combined with at least one additional active agent suitable for acting as an activity enhancer and/or for acting in a complementary manner on one or more other activities.

For this purpose, various additional actives are mentioned below in the detailed description.

According to a third object, a process is also provided for improving the aesthetic appearance of the skin and its appendages comprising the topical application to the skin of an effective amount of a composition comprising at least one peptide according to the invention described above.

In the case of a dermatological composition, the latter will be suitable for an antimicrobial curative treatment (antibacterial and/or antifungal) and/or an anti-inflammatory curative treatment, this, as seen above, thanks to the inhibitory effect of the peptide shown on a growth curve of the Propionibacterium acnes acne bacterium and the strong stimulation of the expression of a large number of anti-microbial peptides (PAM), in particular capable of inhibiting the growth of yeasts of the genus Malassezia responsible for dandruff, thanks to the repair of the cutaneous defense system (anti-inflammatory and immune) against bacteria, oxidants, radiations, and thanks to cutaneous barrier reconstitution and hydration reinforcement.

The present invention therefore also provides according to a fourth object the peptide for a therapeutic treatment comprising the application to a skin in need thereof of an effective amount of the peptide according to the invention, the treatment being in particular antimicrobial (antibacterial or antifungal), and/or anti-inflammatory, said treatment being in particular suitable for soothing sensitive and irritated skin and/or for treating acne, psoriasis, dermatitis and eczema.

According to the invention, the peptide is adapted for use in the preparation of a cosmetic composition for treating acne-prone skin and/or for hydrating and/or smoothing the skin and/or for preventing the appearance of dandruff and/or or protect the skin microbiota and/or strengthen skin immunity.

According to the invention, by “topical treatment” or “topical use” is meant an application which is intended to act at the place where it is applied: skin, mucous membrane, appendages.

The peptide or the composition comprising it according to the invention can be applied locally to the targeted areas.

The “effective” amount depends on various factors, such as the age, condition of the patient, the severity of the disorder and how it is administered. An effective amount means a non-toxic amount sufficient to achieve the desired effect.

In a cosmetic composition according to the invention, the at least one peptide, in order to be present in an effective amount, is generally found in proportions of between 0.01 ppm and 500 ppm relative to the total weight of the composition, preferably between 0.1 ppm and 50 ppm, more preferably between approximately 1 ppm and 10 ppm, depending on the destination of the composition and the desired effect more or less pronounced.

All percentages and ratios used in the present application are by weight of the total composition and all measurements are made at 25° C., unless otherwise specified.

For example, for a cosmetic treatment of the face, the European Cosmetics Directive has set a standard amount of application of a cream of 2.72 mg/cm²/day/person and for a body lotion of 0.5 mg/cm²/day/person.

According to other particularities, the cosmetic treatment method according to the invention can be combined with one or more other treatment methods targeting the skin, such as, for example, treatments by light therapy, by heat, by vibrations, by electroporation, micro-needles patch or by aromatherapy.

According to the invention, it is possible to propose devices with several compartments or kits intended for the implementation of the method described above, and which could comprise, by way of example, and without limitation, in a first compartment a composition comprising one or more peptides according to the invention and in a second compartment an excipient and/or additional active, the compositions contained in said first and second compartments being considered here as a combination composition for simultaneous, separate or spread use in time in particular in one of the treatments defined above.

A composition according to the invention is also suitable for a therapeutic treatment, in particular a treatment of the skin, in particular also of a skin having a diseased epidermis, at suitable doses.

### DETAILED DESCRIPTION

The present invention will be better understood in the light of the description which will follow of an embodiment and of in vitro and in vivo tests.

### A) Example of Preparation of the Pal-KTTKS Peptide (SEQ ID NO 1) According to the Invention

The Pal-KTTKS peptide is prepared by peptide synthesis. A serine is coupled with a resin via its terminal acid function (with a coupling agent, for example DCC (diclyclohexylcarbodiimide)/NHS (N-hydroxysuccinimide) or HBTU (2-(1H-benzotriazol-1-yl)-1, 1, 3, 3-tetramethyluronium hexafluorophosphate)/HOBT (1-hydroxy-benzotriazole)). The serine thus protected is then reacted with a lysine derivative in the presence of a coupling agent, then the same operation is carried out to add two threonines, then the same operation to add the second lysine. The latter is then acylated on its amine function with an activated palmitic acid derivative (palmitoyl chloride for example) in the presence of a base. The peptide chain is cleaved from the resin in an acidic medium and after precipitation, washing and drying. The product palmitoyl-lysyl-threonyl-threonyl-lysyl-serine is obtained in solid form.

### B) Example of Preparation of a Cosmetic Active Ingredient According to the Invention Comprising the Pal-KTTKS (SEQ ID NO 1)

The Pal-KTTKS peptide is amphiphilic, the Pal chain being hydrophobic and the peptide part being hydrophilic. The peptide, for example at 100 ppm, is solubilized in a water/glycol matrix with suitable surfactants.

The Matrixyl® commercial ingredient comprising Pal-KTTKS (Palmitoyl Pentapeptide-4) can be used for implementing the invention.

### C) Efficacy Tests

They were carried out on the preferred peptide according to the invention, the Pal-KTTKS, in solution in an inert solvent (ethanol), at concentrations recommended for use on the skin.

The in vitro tests below were performed on epidermis cells, the normal human keratinocytes (NHK). A test was carried out on a culture of the bacterium Propionibacterium acnes.

The Various Test Methods Used are Described Below.

Molecular Biology Study on NHK Culture

Principle: in this test, confluent keratinocytes are brought into contact with the peptide according to the invention for 24 hours. The cells are then frozen to dryness and their RNAs are extracted, converted into DNA, then analyzed by qRT-PCR (“Quantitative real time reverse transcription polymerase chain reaction”). The results are expressed as an expression ratio between the treated case and the control case. A ratio greater than 1.5 is as an induction of gene expression (+50% compared to the control). A study of variances and a Student's t test are carried out for judging the significance of the results (n=4 for each condition).

Study by Immune Enzymatic Assay on NHK Culture

Principle: NHKs in culture and at confluence are brought into contact with the peptide according to the invention (or its solvent) for 24 hours in a medium allowing their survival. Then the cell layers are irradiated with UVB in a physiological buffer and again brought into contact with the products to be tested for 24 hours. At the end of this incubation, the culture media are assayed by ELISA to know the amounts of pro-inflammatory mediators produced by these cells in response to irradiation. The results are compared to the control. A cell respiration test is conducted on the fixed mat to assess the number of cells and standardize the results. A study of variances and a Student's t test are carried out for judging the significance of the results.

Study Using DNA Microarray Technology (DNA-Array) on NHK Culture

Principle: the peptide according to the invention (7 ppm) is brought into contact for 24 or 48 hours with confluent NHKs (vs. control case). Then the NHK mats are rinsed, and the cells are crushed to extract their mRNA. These mRNAs are then converted into DNA sequences which are analyzed after depositing on DNA chips and amplification by a method like QRT-PCR. The mRNA variations due to the peptide are compared to the control case (peptide solvent). The results are expressed as an expression ratio between the treated case and the control case. A ratio greater than 1.5 is as an induction of gene expression (+50% compared to the control).

Liquid Chromatography/Mass Spectrometry (LC-MS/MS) Study on NHK Culture

Principle: the same culture protocol as for the DNA-Array is used but with a longer culture time (7 days) because the protein productions take longer to be able to be detected with this method in LC-MS/MS. The peptide concentration applied to the cells is 5 ppm (vs. control case). The culture medium of the NHK (n=3) is changed every 3 days. At the end of this contact, the cells are lysed for extracting the proteins and to analyze them in the form of ground material by a method combining the action of a protease on the ground material, the separation of the fragments by liquid chromatography coupled with mass spectrometry, and then the identification and concentration of pre-existing proteins according to the nature and quantity of the fragments obtained (LC-MS/MS). To carry out LC-MS/MS analysis on equivalent amounts of proteins, the protein concentration is measured on the ground material. The results are expressed as an expression ratio between the treated case and the control case. A ratio greater than 1.5 is considered as an increase in protein production (+50% compared to the control). A study of variances and a Student's t test is carried out for judging the significance of the results.

Study by Immunocytofluorescence Labeling on KHN Culture

Principle: confluent NHKs (n=3) are cultured as above for 7 days in the presence or absence (control) of the peptide according to the invention then the cell layers are fixed and labels by immunocytochemistry are carried out using antibodies directed against proteins characteristic of the maturation of the epidermis: involucrine, loricrin and filaggrin. Photos are captured using a fluorescence microscope and images analyzed using appropriate software. The results are compared with those of the control. The number of cells on the mats is evaluated by the Hoechst method (DNA staining), to reduce the fluorescence value to the number of cells. A variance study and a Student's t test are carried to judge the significance of the results.

Propionibacterium acnes Growth Inhibition Test

Principle: suspensions of Propionibacterium acnes of equivalent density are cultured in a suitable medium in the presence (test case) or absence (control case) of the peptide according to the invention, in anaerobic condition, at 37° C. Samples are taken at regular intervals to measure the OD at 600 nm for following the growth of the bacterial population over time. The growth curve of each culture is thus established.

1. Improvement of the Epidermal Barrier and Harmonization of the Maturation of the Epidermis

The cornified layer or stratum corneum is an assembly of great complexity associating, on the one hand, cells without nucleus, flat and strongly linked to each other and, on the other hand, lipids and proteins whose composition and assembly ensure the unique properties of this structure very resistant to physical, chemical and biological attacks from the environment.

The test results given below show that the peptide according to the invention improves epidermis homeostasis and skin barrier reinforcement, thanks to the assay of different markers involved in epidermal differentiation and barrier formation.

The keratinocytes gradually mature by acquiring a very resistant outer shell formed of proteins called involucrine and loricrin, linked together thanks to the intervention of transglutaminases, enzymes sensitive to calcium. In addition, SPRRs (Small Proline Rich Region Proteins), other proteins involved in maturation and homeostasis of the stratum corneum, serve to strengthen this protein shell by creating flexible but resistant bridges between proteins, again thanks to the activity of transglutaminases. There are also the LCEs (“Late Cornified Envelope Proteins”) which are among the last components to be bridged during the maturation phase. Furthermore, ceramides are of great importance in the formation of the stratum corneum and of its proteolipid matrix, hence the importance of cosmetic active ingredients stimulating the synthesis of these elements. A good barrier function is also very dependent on the filaggrins which are produced by the keratinocytes where they undergo significant metabolism. They serve for a time to stabilize the corneocyte by binding to the keratins and then end up being broken down into amino acids giving essential components of the natural hydration factor (NMF) found in the stratum corneum, which allows a good hydration of the skin

Furthermore, kallikreins are proteases involved in the renewal of the stratum corneum by allowing natural desquamation, thus ensuring a gentle “natural” smoothing effect. The increased expression and synthesis of these enzymes improves the natural process of corneocyte desquamation and may be similar to a natural peel.

1.1. Action on Epidermal Differentiation and Stratum Corneum Formation

1.1.1. Induction of the formation of involucrin, loricrin and filaggrin in NEM (by immunofluorescence)/effect of 5 ppm of the peptide according to the invention:

TABLE 1

| | Involucrin: | Loricrin: | Filaggrin: |
|---|---|---|---|
| | % of variation/ | % of variation/ | % of variation/ |
| Concentrations | control | control | control |
| Control | Reference | Reference | Reference |
| 5 ppm | +1057%; p < 0.05 | +128%; p < 0.01 | +310%; p < 0.01 |

1.1.2. Variation compared to the control of the expression of genes (DNA-Array) coding for proteins of epidermal differentiation and formation of the stratum corneum/effect of 7 ppm of the peptide according to the invention at 24/48 hours of contact:

TABLE 2

| Expressed | | |
|---|---|---|
| gene | Variation | Name and function of the protein |
| PRSS8 | ×3.72 | Serine protease 8; cleaves profilaggrin into filaggrin. |
| ASPRV1 | ×3.35 | Skin aspartic protease; cleaves profilaggrin into filaggrin. |
| LOR | ×1.86 | Loricrin; major protein of the stratum corneum which confers to |
| | | it cohesion/rigidity; linked to the other proteins of the envelope by |
| | | the enzymatic action of TGM1 (see below). |
| TGM1 | ×1.70 | Transglutaminase 1; enzyme that cross-links between different |
| | | structural proteins, at the level of the homy envelope, to ensure its |
| | | rigidity. |
| SPRR2A | ×8.71 | Small Proline Rich Region Proteins; maturation and homeostasis |
| SPRR2C | ×6.30 | proteins of the stratum corneum, serve to strengthen the protein |
| SPRR2E | ×6.47 | shell of the corneocyte by creating flexible but resistant bridges |
| SPRR3 | ×1.90 | between proteins, here again thanks to the activity of |
| SPRR2F | ×6.00 | transglutaminases |
| SPRR2D | ×7.87 | |
| SPRR2B | ×58.44 | |
| SPRR2G | ×13.43 | |
| SPRR4 | ×2.95 | |
| LCE3A | ×65.26 | Late Cornified Envelop Proteins; these are the last components to |
| LCE3B | ×123.00 | be bridged during the maturation phase in the stratum corneum. |
| LCE3D | ×110.46 | |
| LCE3E | ×135.99 | |
| LCE2C | ×26.30 | |
| CRNN | ×4.73 | Cornulin; marker of terminal epidermal differentiation. |
| | (48 h) | |
| TCHH | ×4.87 | Trichohyalin; structural protein that helps strengthen the barrier |
| | (48 h) | by cross-bridging with other proteins in the stratum corneum |
| | | using the enzyme TGM1. |
| BMP6 | ×13 | Bone morphogenetic protein 6; regulates positively keratinocyte |
| | | differentiation (including expression of keratin 1) through |
| | | multiple signaling systems. |

1.1.3. Variation compared to the control of proteins related to the maturation of the epidermis (by LC-MS/MS)/effect of 5 ppm of the peptide according to the invention:

TABLE 3

| Protein | Variation | Name and function of the protéine |
|---|---|---|
| FLG | ×2.66; p < 0.01 | Filaggrin; as disclosed above |
| LOR | ×2.92; p < 0.01 | Loricrin; as disclosed above |
| ASPRV1 | ×2.35; p < 0.01 | Skin aspartic protease; cleaves profilaggrin into filaggrin. |
| CALM5 | ×1.61; p < 0.01 | Calmodulin 5; forms a complex with calcium; calmodulins |
| | | control many enzymes, ion channels, aquaporins and other |
| | | proteins through their binding with calcium. |
| TCHH | ×1.54; p < 0.01 | Trichohyalin; as disclosed above |
| KRT1 | ×1.58; p < 0.01 | Keratins; main epidermis constituent, at the stratum corneum |
| KRT9 | ×1.54; p < 0.01 | level; form an intracellular network; in the form of filaments |
| KRT10 | ×1.75; p < 0.01 | sheathed by a shell of strongly crosslinked proteins and are |
| KRT15 | ×2.02; p < 0.01 | connected to the desmosomes. |

1.1.4. Variation compared to the control of the gene expression of involucrin and transglutaminase 1 to 24 hours (by RT-PCR)/effect of the peptide according to the invention at 5 ppm:

TABLE 4

| Expressed | | |
|---|---|---|
| gene | Variation | Name and function of the protein |
| Involucrine | ×1.64; p < 0.01 | Involucrin; major protein of the |
| | | stratum corneum which confers its |
| | | cohesion/rigidity; linked to other |
| | | envelope proteins by the enzymatic |
| | | action of TGM1 (see above) |
| Transglutaminase 1 | ×1.82; p < 0.05 | As disclosed above |

1.2. Action on the Formation of Lipidic Lamellar Bodies

Variation compared to the control of the expression of genes (DNA-Array) encoding proteins involved in the formation of the lipid envelope of the corneocyte/effect of 7 ppm of the peptide according to the invention at 24 or 48 hours of contact:

TABLE 5

| Expressed | | |
|---|---|---|
| gene | Variation | Name and function of the protein |
| ALOX12B | ×2.48 | Arachidonate 12-lipoxygenase; acts upstream of ALOXE3 on |
| | | the lineolate fragment of esterified omega-hydroxyacyl- |
| | | sphingosin ceramides to produce an epoxy-ketone derivative, |
| | | a crucial step in the conjugation of omega-hydroxyceramide to |
| | | membrane proteins; therefore plays a crucial role in the |
| | | synthesis of the lipid envelope of corneocytes and in the |
| | | establishment of the skin barrier. |
| ALOXE3 | ×2.92 | Hydroperoxide isomerase; acts downstream of ALOX12B on |
| | | the linoleate moiety of esterified omega-hydroxyacyl- |
| | | sphingosine (EOS) ceramides to produce an epoxy-ketone |
| | | derivative, a crucial step in the conjugation of omega- |
| | | hydroxyceramide to membrane proteins; therefore plays a |
| | | crucial role in the synthesis of the lipid envelope of |
| | | corneocytes and in the establishment of the skin barrier. |
| CERS1 | ×3.48 | Ceramide synthase 1; catalyzes the formation of ceramides |
| | (48 h) | from sphinganine substrates and acyl-CoA. |
| ELOVL4 | ×5.41 | 3-keto acyl-CoA synthase; catalyzes the limiting step of the 4 |
| | | reactions of the cycle of elongation of long chain fatty acids; |
| | | formation of the corneocyte lipid envelope. |
| ABHD5 | ×2.10 | 1-acylglycerol-3-phosphate O-acyltransferase; this enzyme |
| | | plays a crucial role in lipid metabolism within the lamellar |
| | | bodies, contributing to the formation of the skin lipid barrier. |

1.3. Action on the Formation of Desmosomes, Corneodesmosomes and Tight Junctions

Variation compared to the control of the expression of genes (DNA-Array) encoding proteins involved in the formation of desmosomes, corneodesmosomes and tight junctions/effect of 7 ppm of the peptide according to the invention at 24 hours of contact:

TABLE 6

| Expressed | | |
|---|---|---|
| gene | Variation | Name and function of the protein |
| CDSN | ×3.36 | Coneodesmosin; presence essential for the integrity of the |
| | | epidermal barrier; protein specific to corneodesmosomes for |
| | | promoting their adhesion. |
| OCLN (*) | ×5.91 | Occludin; plays an important role in the formation and |
| | | regulation of tight junction; capable to induce adhesion when |
| | | expressed in cells lacking tight junctions. |
| TJP1 | ×2.15 | Tight junction protein ZO-1; structural protein connecting |
| | | tight junction transmembrane proteins such as claudins and |
| | | occludin to the actin cytoskeleton. |
| CGNL1 | ×3.37 | Cingulin-like protein 1; involved in the anchoring of tight |
| | | junctions to the actin cytoskeleton. |
| CLDN4 | ×5.16 | Claudins; proteins present at the level of the tight junctions |
| CLDN7 | ×4.47 | which ensure cohesion between the corneocytes, via the actin |
| CLDN12 | ×2.68 | network in the upper part of the epidermis; they therefore |
| CLDN15 | ×4.80 | have a guardian role for water homeostasis, preventing the |
| CLDN17 | ×113.20 | evaporation of water. |
| CLDN23 | ×3.69 | |
| DEFB103B | Very | Human Beta defensin 3; improves the barrier function via the |
| | strongly | tight junctions, by the CCR6 receptor, but also by a strong |
| | expressed | induction of claudins (see in this tabic), aPKC kinases, Rac1 |
| | | essential in the regulation of tight junctions, GSK3 involved |
| | | in the expression of tight junction proteins. |

(*) effect confirmed by RT-PCR (×3.03 for 5 ppm of PalTTTKS at 24 hours of contact)

1.4. Desquamation Regulation

Variation compared to the control of the expression of genes (DNA-Array) encoding proteins involved in the regulation of desquamation/effect of 7 ppm of the peptide according to the invention at 24 hours of contact:

TABLE 7

| Expressed | | |
|---|---|---|
| gene | Variation | Name and function of the protein |
| KLK14 | ×1.79 | Kallikrein-14; serine protease which cleaves |
| | | desmoglein during epidermal desquamation |
| | | (corneocytcs excreted from the surface of the |
| | | skin). |

Conclusion: It appears that the peptide according to the invention acts at all the levels studied: from the metabolization of filaggrin to the regulation of desquamation, including the production of elements of the stratum corneum, the formation of lipid lamellar bodies, the formation of tight junctions in a way that improves and strengthens the epidermal barrier.

The peptide according to the invention thus acts favorably to guarantee the homeostasis of the epidermis, ensuring a good balance between the renewal by differentiation of the cells and the formation of a skin barrier of effective quality in particular with regard to external attacks. This type of activity profile guarantees good re-epithelialization of the skin tissue in subjects who have experienced an acne episode, by reducing unaesthetic acne scars.

2. Skin Protection Action Including Defense Against Bacteria, Inflammation, Radiation and Immunity Stimulation

2.1. Human Beta Defensin 3 (HBD3) and Other Markers

HBD3 is an antimicrobial peptide that intervenes at several levels. It is a key molecule in the skin's immune system. In particular, it has a broad spectrum of destruction against bacteria and yeasts. Thus, stimulating the expression of this peptide has a great interest in cosmetics, on the one hand in the prevention and treatment of acne (against the Propionibacterium acnes bacterium) and on the other hand to treat a dandruff condition (against yeasts of the genus Malassezia). Recently, it was found that these antimicrobial defense peptides have a broader action in the skin and that they have a role in cell proliferation, migration and differentiation, in the regulation of inflammatory responses, by controlling the production of different cytokines, in the development of cicatrisation in the epidermis, and in improving the barrier function.

The peptide according to the invention has an anti-inflammatory role demonstrated by the DNA-Array with the induction of the anti-inflammatory cytokine IL-37. It is further described as involved in skin immunity via the production of various cytokines/chemokins. Furthermore, it is also described to counter the inflammatory effects of bacterial lipopolysaccharide. Finally, it has feedback control of inflammatory activity by inhibiting the TLR (Toll Like receptor) pathway.

Thus, the peptide according to the invention, comprised a cosmetic composition, by being capable of strongly stimulating the gene expression of human beta defensin 3, an integral part of the innate immune system, will have a defensive action to prevent penetration or proliferation of infectious agents in the skin. It is an immediate response, in the form of inflammatory reactions, not specific to the pathogen agent.

The peptide according to the invention also stimulates the expression of the SOD2 gene, important in the defense of the skin against free radicals (here the superoxide radical).

A direct action against the bacteria Propionibacterium acnes enhances the interest of the peptide according to the invention for fighting acne.

Results

Variation compared to the control of the expression of genes (DNA-Array) encoding proteins involved in the protection of the skin/effect of 7 ppm of the peptide according to the invention at 24 or 48 hours of contact:

TABLE 8

| Expressed | | |
|---|---|---|
| gene | Variation | Name and function of the protein |
| DEFB103B | Very strongly | Human Beta Defensin 3; as disclosed |
| | expressed | above |
| SOD2 | ×2.93 | Superoxide dismutase 2; destroys the |
| | (48 hours) | superoxide anion radicals normally |
| | | produced in cells and which are toxic |
| | | to biological systems. |
| IL37 | ×3.60 | Interleukin-37; suppresses or reduces the |
| | | production of pro-inflammatory cytokins, |
| | | including IL1a and IL6. |

Variation compared to the control of proteins involved in the protection of the skin (by LC-MS/MS)/effect of 5 ppm of the peptide according to the invention:

TABLE 9

| Expressed | | |
|---|---|---|
| gene | Variation | Name and function of the protein |
| SPINK5 | ×1.58; p < 0.01 | Serine protease inhibitor Kazal-type 5; |
| | | serine protease inhibitor, important for |
| | | anti-inflammatory and/or antimicrobial |
| | | protection of the skin; contributes to the |
| | | integrity and barrier function of the skin |
| | | by regulating the activity of proteases |
| | | involved in the desquamation and defense |
| | | of the skin. |

2.2. Specific Action Against Propionibacterium acnes

Growth of the bacterium Propionibacterium acnes: time necessary to reach an OD of 1 in the control case and in the presence of the peptide according to the invention (N=2 independent experiments; n=2 cultures per case):

TABLE 10

| | | 6 ppm of | 9 ppm of | 12 ppm of |
|---|---|---|---|---|
| Cases | Control | peptide | peptide | peptide |
| Time in hours | 28 | 42 | 54 | 85.5 |

These data show that the peptide according to the invention strongly and dose-dependently inhibits the growth of Propionibacterium acnes.

2.3. Alpha Crystallin

Alpha-crystallin is a small protein related to the HSP family (or heat shock proteins). It is present in the epidermis, where it protects epithelial cells, restores their mitochondrial functions, and increases their resistance to oxidative stress. This protein has the property of being found in the cell and in its immediate vicinity. Therefore, the skin is protected from UV, inflammatory and more generally oxidative attacks by its presence.

Variation compared to the control of the expression of the alpha-crystallin B gene 1 to 24 hours (by RT-PCR)/effect of the peptide according to the invention at 5 ppm:

TABLE 11

| Expressed | | |
|---|---|---|
| gene | Variation | Name and function of the protein |
| CRYAB | ×3.47; p < 0.01 | Alpha-crystallin B; as disclosed above |

Variation compared to the control of proteins involved in the defense of the skin (by LC-MS/MS)/effect of 5 ppm of the peptide according to the invention:

TABLE 12

| Expressed | | |
|---|---|---|
| gene | Variation | Name and function of the protein |
| CRYAB | ×2.24; p < 0.01 | Alpha-crystallin B; as disclosed above |

2.4. Moderation of the Production of Inflammation Markers

The skin is subjected to constant stress (exposure to UV rays, smoke, pollutants, etc.) some of which provoke a direct or indirect inflammatory response. The uncontrolled or constant inflammatory response, although of low intensity, leads to the production of cytokines such as IL-1α, IL-1β, IL-6, TNF α, and lipids such as PGE2 intended to attract or stimulate other cells, causing cascading reactions. The pro-inflammatory microenvironment thus formed leads to modify the homeostasis of the skin and gradually leads to the modification or even destruction of the biomolecules of cells and tissues. It also leads to the disruption of the skin barrier integrity. Thus, the mediators of inflammation, IL-6 and PGE-2, are known to induce, via micro-inflammations, the phenomena of premature aging. In addition, sensitive and irritated skin is characterized by an abnormally high secretion of cytokines, pro-inflammatory peptides (IL-1, IL-6 for example) and pro-inflammatory lipids (PGE-2 for example).

To test the active ingredients, keratinocytes were placed in culture under light stress conditions (application of UVB radiation) to mimic an experimental skin micro-inflammation. In this situation, a significant decrease in their secretome of inflammation mediators will be interpreted in the sense of a restorative and protective action of the skin.

Results:

Variation compared to the control of the secretome of pro-inflammatory mediators by NHKs exposed to UVB (by immunoenzymatic assay)/effect of the peptide according to the invention at 4, 6 and 8 ppm:

TABLE 13

| Marker | 4 ppm | | 6 ppm | 8 ppm |
|---|---|---|---|---|
| IL6 | −13% | (ns) | −68% (p < 0.01) | −84% (p < 0.01) |
| PGE2 | −41% | (p < 0.01) | −69% (p < 0.01) | −84% (p < 0.01) |

ns: non-significant

These results show the very interesting effect of the peptide according to the invention for moderating the secretion of pro-inflammatory mediators by NHKs which have been exposed to UVB irradiation. This effect is particularly interesting for sensitive skin irritated by exposure to radiations and various pollutants that cause micro-inflammations.

To these results is also added the expression of the IL37 gene mentioned above.

Conclusion:

The peptide according to the invention regulates in the sense of an enhanced defense of the skin against bacteria, oxidants, radiation and against the resulting inflammation, all the markers studied acting toward this aim.

3. Action to Prevent and Treat Scars on the Epidermis

Human Beta Defensin 3 participates in cicatrisation by promoting keratinocyte migration and proliferation. These actions imply phosphorylation induction of EGFR, a signal transducer and of STAT1 and STAT3, intracellular signaling molecules known to be involved in keratinocyte migration and proliferation.

Certain tight junction (TJ) proteins, such as occludin, are also involved in keratinocyte migration and proliferation. These processes are essential for the healing of normal skin wounds and the regeneration of the epidermis.

The signaling pathways of BMPs, members of the TGFβ superfamily, regulate the healing process by directing keratinocytes either in the proliferative pathway or towards differentiation, depending on the stage of healing.

Variation compared to the control of the expression of genes (DNA-Array) coding for proteins involved in cicatrising/effect of 7 ppm of the peptide according to the invention at 24 or 48 hours of contact:

TABLE 14

| Expressed | | |
|---|---|---|
| gene | Variation | Function of the protein |
| DEFB103B | Very strongly | Human Beta defensine 3; as disclosed above |
| | expressed | |
| ADGRG1 | ×1.92 | Receptor 56 coupled to G protein; involved in cell |
| | | adhesion and probably in cell-cell interactions. |
| FN1 | ×3.38 | Fibronectin 1; fibronectins bind to the surface of cells. |
| | | They are involved in cell adhesion, cell motility, wound |
| | | healing, and the maintenance of cell shape. |
| GSK3B | ×1.64 | Glycogène synthase kinase 3 beta; GSK-3beta controls |
| | | the progression of wound healing by modulating |
| | | endothelin-1 levels. |
| BMP2 | ×3.63 | Bone morphogenetic protein 2; as disclosed above |
| BMP6 | ×13 | Bone morphogenetic protein 6; as diclosed above |
| OCLN (*) | ×5.91 | Occludin; as disclosed above |
| PPARD | ×1.97 | Peroxisome proliferator activated receptor delta; |
| | | transcription factor involved in epidermal |
| | | differentiation, maintenance of epidennal homeostasis |
| | | and anti-inflammatory response. |

(*) effect confirmed by RT-PCR (×3.03 for 5 ppm of PalKTTKS at 24 hours of contact)

Variation compared to the control of proteins involved in healing (assay by LC-MS MS)/effect of 5 ppm of the peptide according to the invention:

TABLE 15

| Protein | Variation | Name and function of the protein |
|---|---|---|
| THBS1 | ×1.98; | Thrombospondin-1; An adhesive glycoprotein |
| | p < 0.01 | that is involved in cell-to-cell and cell-to- |
| | | matrix interactions. |
| ADGRG1 | ×1.63; | Receptor 56 coupled to G protein; as disclosed |
| | p < 0.01 | above |
| KRT6 | ×1.47; | Keratin 6; induced during healing, at the |
| | p < 0.01 | suprabasal level. |

Conclusion:

All the studied markers show that the peptide according to the invention acts in the keratinocyte level by improving the healing process.

4. Beneficial Action on Nails and Hair

4.1. On Nails

The BMPs, members of the TGFβ superfamily, are involved in nail differentiation and development. They promote communication between the epidermis and the mesenchyme, and coordinate differentiation, by activating transcription factors.

Variation compared to the control of the expression of genes (DNA-Array) encoding constituent proteins of the nail or involved in its formation/effect of 7 ppm of the peptide according to the invention at 24 or 48 hours of contact:

TABLE 16

| Gene | Variation | Name and function of the protein |
|---|---|---|
| TCHH | ×4.87 | Trichohyalin; present in the nail bed |
| | (48 hours) | |
| BMP2 | ×3.63 | Bone morphogenetic protein 2; as disclosed above |
| BMP6 | ×13 | Bone morphogenetic protein 6; as disclosed above |

Variation compared to the control of constituent proteins of the nail or involved in its formation (assay by LC-MS/MS). Effect of 5 ppm of the peptide according to the invention.

TABLE 17

| Protein | Variation | Name and function of the protein |
|---|---|---|
| KRT6 | ×1.47; p < 0.01 | Keratinc 6; present in the nail bed |
| TCHH | ×1.54; p < 0.01 | Trichohyaline; present in the nail bed |
| KRT 1 | ×1.58; p < 0.01 | Keratine 1; present in the suprabasal matrix, |
| | | absent from the bed |
| KRT 10 | ×1.75; p < 0.01 | Keratine 10; present in the suprabasal matrix, |
| | | absent from the bed |
| FLG | ×2.66; p < 0.01 | Filaggrine; present in the nail bed |

Conclusion:

Several compounds of the nail itself and of compounds involved in its formation have their gene expression and/or their concentration increased following contact with the peptide according to the invention. This was observed in the DNA-Array and in the LC-MS/MS protein study.

The compound according to the invention thus appears to be perfectly indicated for the maintenance of healthy nails or the treatment of damaged nails.

4.2. On Hair

Morphogenesis of hair follicles and initiation of a growth phase of quiescent hair follicles, are characterized by the activation of different signaling pathways resulting in the construction of a hair shaft. Among the different signaling pathways, the balance between the activities of the Wnt/β-catenin and BMP pathways is particularly important.

BMPs therefore participate in the regulation of hair growth by inhibiting the proliferation phase of dermal papilla cells and stimulating the differentiation of cells synthesizing the hair shaft. Conversely, the Wnt/β-catenin pathway results in the activation of proliferation of the dermal papilla cells.

Trichohyalin is expressed in specific epithelia, exceptionally strong mechanically, such as the cells in the inner sheath of the hair follicle. It is subjected to modifications by enzymes, in particular transglutaminases, which introduce intra- and inter-proteic cross-links. It is a multifunctional protein cross-linked by bridges that functions inside the hair's inner root sheath, imparting and coordinating mechanical strength between peripheral cell envelope structures and the cytoplasmic keratin filament network.

Variation compared to the control of the expression of genes (DNA-Array) encoding constituent proteins of the hair or involved in its formation/effect of 7 ppm of the peptide according to the invention at 24 or 48 hours of contact:

TABLE 18

| Expressed | | |
|---|---|---|
| gene | Variation | Function of the protein |
| TCHH | ×4.87 | Trichohyalin; as disclosed above |
| | (48 hours) | |
| BMP2 | ×3.63 | Bone morphogenetic protein 2; as disclosed above |
| BMP6 | ×13 | Bone morphogenetic protein 6; as disclosed above |
| GSKA | ×1.57 | Glycogene synthase kinase 3 alpha; regulates hair |
| | | growth |
| GSKB | ×1.64 | Glycogene synthase kinase 3 beta; key enzyme in |
| | | the Wnt/β-catenin signaling pathway that |
| | | regulates hair growth. |

Variation compared to the control of proteins constituting the hair or involved in its formation (assay by LC-MS/MS)/effect of 5 ppm of the peptide according to the invention:

TABLE 19

| Expressed | | |
|---|---|---|
| gene | Variation | Function of the protein |
| KRT6 | ×1.47; p < 0.01 | Keratin 6; expressed in the outer sheath |
| | | of the hair follicle. |
| KRT6B | ×2.07; p < 0.01 | Keratin 6B; constitutively expressed |
| TCHH | ×1.54; p < 0.01 | Trichohyalin; as disclosed above |

Conclusion:

A certain number of compounds of the hair itself and of compounds involved in its formation have their gene expression and/or their concentration increased following contact with the peptide according to the invention. This was observed in the DNA-Array and in the LC-MS/MS protein study.

The compound according to the invention thus appears to be perfectly indicated for an action in the hair field to improve the strength and growth of the hair.

### D) Galenics/Preparation of a Composition According to the Invention

The peptide according to the invention can be formulated with additional cosmetic active ingredients, that can come in support and/or in complement of activity, either in the ingredient form, or at the time of the realization of the final cosmetic composition for the consumer. This composition can be applied to the face, body, neckline, scalp, hair, eyelashes, body hair, in any form or vehicles known to those skilled in the art, in particular in the form of solution, dispersion, emulsion, paste or powder, individually or as a premix or be conveyed individually or as a premix.

In cosmetics, applications can be proposed in particular in the ranges of skin care for the face, body, hair and body hair and ranges of make-up treatments.

These ingredients can be of any category depending on their function(s), the place of application (body, face, neck, bust, hands, hair, eyelashes, eyebrows, body hair, etc.), the desired final effect and the targeted consumer, for example antioxidant, tensor, moisturizer, nourishing, protective, smoothing, remodeling, volumizing (lipofiling), acting on the radiance of the complexion, anti-dark spots, concealer, anti-glycation, anti-aging, anti-wrinkle, slimming, soothing, myo-relaxing, anti-redness, anti-stretch marks, sunscreen, etc.

The CTFA (“International Cosmetic Ingredient Dictionary & Handbook” (19th Edition, 2019) published by “The Cosmetic, Toiletry, and Fragrance Association, Inc.”, Washington, D.C.) describes a wide variety, without limitation, of cosmetic ingredients usually used in the skincare industry, which are suitable for use as additional ingredients in the compositions of the present invention.

At least one of the compounds can be cited selected from vitamin B3 compounds, compounds such as niacinamide or tocopherol, retinoid compounds such as retinol, hexamidine, α-lipoic acid, resveratrol or DHEA, hyaluronic acid, peptides, in particular N-aceyl-Tyr-Arg-O-hexadecyl ester, Pal-VGVAPG (SEQ ID NO 3), Pal-KTFK (SEQ ID NO 6), Pal-GHK, Pal-KMO₂K, Pal-GQPR (SEQ ID NO 2), Pal-K(P)HG (with a proline grafted on the lysine), and Pal-KTSKS (SEQ ID NO 7), which are known active ingredients used in topical cosmetic or dermo-pharmaceutical compositions.

Other additional skin care actives that are particularly useful can be found in Sederma's commercial literature and at www.sederma.com or www.crodarom.com.

For reinforcing the activity on the properties of the epidermis and/or stratum corneum, the additional active agent can be chosen from the group comprising: phospholipids, various ceramides, sphingosine, phytosphingosine, glycosphingolipids, cholesterol and its derivatives, sterols (in particular those of canola and soybean), fatty acids (in particular linoleic acid, palmitic acid, lipoic acid, thioctic acid), squalane (in particular olives), triglycerides (especially coconut oil), lanolin, lanolin alcohols, lanosterol, vitamin D3, tocopheryl nicotinate, various oils (in particular, argan, rose, baobab), ascorbic acid, N-acetyl cysteine and N-acetyl-L-serine, vitamin B3 compounds (such as niacinamide and nicotinic acid), panthenol, pseudofilaggrin, arginine, serine, salts of PCA (pyrrolidone carboxylic acid) an extract of Centella asiatica leaf (titrated in madecasso side and asiaticoside), certain plant extracts (wild yam roots, chestnut, cedar bud, nightshade), plankton and yeast.

Mention may also be made of the active agents marketed by Sederma: Venuceane™ (extract of fermentation medium of Thermus thermophilus), Moist24™ (hydroglycolic extract of Imperata cylindrica root), Dermaxyl™ (combination of ceramide 2 and the Pal-VGVAPG peptide), Senestem™ (a cell culture extract of Plantago lanceolata), Ceramide 2™ (ceramide), Ceramide HO3™ (hydroxyceramide), Optim Hyal™ (acetylated glucuronic acid oligosaccharides), Meiritage™ (combination of root extracts of Bupleurum falcatum, Astragalus membranaceus, Atractylodes macrocephala), Revidrat™ (myristyl phosphomalate), Pacifeel™ (an extract of Mirabilis jalapa), Hydronesis™ (resulting from the fermentation of Salinococcus hispanicus), NG unsaponifiable Shea™ and Citystem™ (a cell culture extract of Marrubium vulgare). The following commercial actives can also be mentioned as examples: betain, glycerol, Actimoist Bio 2™ (Active organics), AquaCacteen™ (Mibelle AG Cosmetics), Aquaphyline™ (Silab), AquaregulK™ (Solabia), Carciline™ (Greentech), Codiavelane™ (Biotech Marine), Dermaflux™ (Arch Chemicals, Inc), Hydra′Flow™ (Sochibo), Hydromoist L™ (Symrise), RenovHyal™ (Soliance), Seamoss™ (Biotech Marine), Argireline™ (commercial name of acetyl hexapeptide-3 from Lipotec), spilanthol or an extract of Acmella oleracea known under the trade name Gatuline Expression™, an extract of Boswellia serrata known under the name Boswellin™, Deepaline PVB™ (Seppic), Syn-AKE™ (Pentapharm), Ameliox™, Bioxilift™ (Silab), PhytoCellTec™ Argan (Mibelle), Papilactyl D™ (Silab), Preventhelia™ (Lipotec), or one or more of the following active ingredient sold by Sederma: Subliskin™, Venuceane™, Moist24™, Vegesome Moist24™, Essenskin™, Juvinity™ Revidrat™, Resistem™, Chronodyn™, Kombuchka™, Chromocare™, Calmosensine™, Glycokin factor S™, Biobustyl™, Idealift™, Ceramide 2™, Ceramide A2™, Ceramide HO3™, Legance™ Intenslim™, Prodizia™, Beautifeye™, Pacifeel™, Zingerslim™, Meiritage™, Senestem™, Sebuless™, Majestem™, Apiscalp™, Rubistem™, Citystem™, Neonyca™, NG Insaponifiables de Beurre de Karité™, Majestem™, Hydronesis™, Poretect™, Crystalide™, Amberstem™, Synchrolife™, Feminage™, or mixture thereof.

Among plant extracts (in the form of classical plant extracts or prepared by an in vitro process) that can be combined with plant cells of Buddleja davidii Franch. according to the present invention, there may more particularly be mentioned extracts of Ivy, in particular English Ivy (Hedera helix), of Bupleurum chinensis, of Bupleurum falcatum, of arnica (Arnica montana L), of rosemary (Rosmarinus officinalis N), of marigold (Calendula officinalis), of sage (Salvia officinalis L), of ginseng (Panax ginseng), of Gingko biloba, of St.-John's-Wort (Hyperycum perforatum), of butcher's-broom (Ruscus aculeatus L), of European meadowsweet (Filipendula ulmaria L), of big-flowered Jarva tea (Orthosiphon stamincus benth), of artichoke (Cynara scolymus), of algae (Fucus vesiculosus), of birch (Betula alba), of green tea, of cola nuts (Cola nipida), of horse-chestnut, of bamboo, of Centella asiatica, of heather, of fucus, of willow, of mouse-ear, of escine, of cangzhu, of Chrysanthellum indicum, of the plants of the Armeniacea genus, Atractylodis platicodon, Sinnomenum, Pharbitidis, Flemingia, of Coleus such as C. Forskohlii, C. blumei, C. esquirolii, C. scutellaroides, C. xanthantus and C. Barbatus, such as the extract of root of Coleus barbatus, extracts of Ballote, of Guioa, of Davallia, of Terminalia, of Barringtonia, of Trema, of Antirobia, Cecropia, Argania, Dioscoreae such as Dioscorea opposita or Mexican, extracts of Ammi visnaga, of Siegesbeckia, in particular Siegesbeckia orientalis, vegetable extracts of the family of Ericaceae, in particular bilberry extracts (Vaccinium angustifollium) or Arctostaphylos uva ursi, aloe vera, plant containing sterols (e.g., phytosterol), Manjistha (extracted from plants of the genus Rubia, particularly Rubia cordifolia), and Guggal (extracted from plants of the genus Commiphora, particularly Commiphora mukul), kola extract, chamomile, red clover extract, Piper methysticum extract (Kava Kava™ from Sederma), Bacopa monieri extract (Bacocalmine™ from Sederma) and sea whip extract, extracts of Glycyrrhiza glabra, of mulberry, of melaleuca (tea tree), of Larrea divaricata, of Rabdosia rubescens, of Euglena gracilis, of Fibraurea recisa Hirudinea, of Chaparral Sorghum, of sun flower extract, of Enantia chlorantha, of Mitracarpe of Spermacocea genus, of Buchu barosma, of Lawsonia inermis L., of Adiantium capillus-veneris L., of Chelidonium majus, of Luffa cylindrica, of Japanese Mandarin (Citrus reticulata Blanco var. unshiu), of Camelia sinensis, of Imperata cylindrica, of Glaucium flavum, of Cupressus sempervirens, of Polygonatum multiflorum, of Loveyly hemsleya, of Sambucus nigra, of Phaseolus lunatus, of Centaurium, of Macrocystis pyrifera, of Turnera diffusa, of Anemarrhena asphodeloides, of Portulaca pilosa, of Humulus lupulus, of Coffea arabica, of Ilex paraguariensis, or of Globularia cordifolia, of Albizzia julibrissin, of Oxydendron arboretum, of Zingimber zerumbet smith, of Astragalus membranaceus, of Atractylodes macrocephala, of Plantago lanceolata, of Leontopodium alpinum, of Mirabilis jalapa, of Apium graveolens, of Marrubium vulgare, Buddleja davidii Franch, Engelhardia chrysolepis, Syringa vulgaris or orchids.

The compositions of the present invention may include one or more additional peptides, including, without limitation, di-, tri-, tetra-, penta- and hexapeptides and their derivatives. According to a particular embodiment, the concentration of the additional peptide, in the composition, ranges from 1×10⁻⁷% and 20%, preferably from 1×10⁻⁶% and 10%, preferably between 1×10⁻⁵% and 5% by weight. The term “peptide” refers here to peptides containing 10 amino acids or less, their derivatives, isomers and complexes with other species such as a metal ion (e.g. copper, zinc, manganese, magnesium, and others). The term “peptides” refers to both natural peptides and synthetic peptides. It also refers to compositions that contain peptides and which are found in nature, and/or are commercially available.

Suitable dipeptides for use herein include but are not limited to Carnosine (βAH), YR, VW, NF, DF, KT, KC, CK, KP, KK, TT, PA, PM or PP.

Suitable tripeptides for use herein include, but are not limited to RKR, HGG, GKH, GHK, GGH, GHG, KGH, KHG, KFK, KAvaK, KβAK, KAbuK, KAcaK, KPK, KMOK, KMO₂K (MO₂ being a di-oxygenated sulfoxide methionine), KVK, PPL, PPR, SPR, QPA, LPA, SPA, K(Ac)HG or K(Ac)GH, K(Ac) being a lysine with the amine function of the lateral chain acetylated, as disclosed in WO2017/216177, K(P)HG or K(P)GH, K(P) being a lysine with its lateral chain grafted with a proline, K(Pyr)HG or K(Pyr)GH, K(Pyr) being a lysine with its lateral chain grafted with a pyroglutamic acid, K(Hyp)HG or K(Hyp)GH, K(Hyp) being a lysine with its lateral chain grafted with a hydroxyproline, as disclosed in WO2016/097965.

Suitable tetrapeptides for use as additional peptides herein include but are not limited to RSRK (SEQ ID NO: 8), GQPR (SEQ ID NO: 9), KTFK (SEQ ID NO: 10), KTAK (SEQ ID NO: 11), KAYK (SEQ ID NO: 12) or KFYK (SEQ ID NO: 13).

A suitable non limitative example of pentapeptide is the KTSKS (SEQ ID NO: 14), and suitable examples of hexapeptides are the GKTTKS (SEQ ID NO: 15), GKTSKS (SEQ ID NO: 16) and VGVAPG (SEQ ID NO: 17).

Other suitable peptides for use according to the present invention can be selected, this list being not limitative, from: lipophilic derivatives of peptides, preferably palmitoyl (Pal) derivatives or myristoyl (Myr), and metal complexes as aforementioned (e.g. copper complex of the tripeptide HGG or GHK). Preferred dipeptides include for example N-Palmitoyl-β-Ala-His, N-Acetyl-Tyr-Arg-hexadecylester (Calmosensine™, Idealift™ from Sederma), Pal-RT or Pal-KT (from Sederma).

Preferred tripeptide derivatives include for example Pal-GKH and Pal-GHK (from Sederma), the copper derivative of HGG (Lamin™ from Sigma), Lipospondin (N-Elaidoyl-KFK) and its analogs of conservative substitution, N-Acetyl-RKR-NH₂ (Peptide CK+), N-Biot-GHK (from Sederma), Pal-KAvaK, Pal-KβAlaK, Pal-KAbuK, Pal-KAcaK, or Pal-KMO₂K (Matrixyl® synthe'6® from Sederma), Pal-KVK (Syn-Coll™ of DSM), and derivatives thereof.

Mention may also be made here of the anti-aging tripeptides of general Formula X-Pro*-Pro*-Xaa-Y described in WO2015181688 application with Xaa selected from Leu, Arg, Lys, Ala, Ser, and Asp, at the N-terminus, X chosen from H, —CO—R¹ and —SO₂—R¹ and at the C-terminal end Y chosen from OH, OR¹, NH₂, NHR¹ or NR¹R², R¹ and R² being, independently of one another, chosen from a alkyl, aryl, aralkyl, alkylaryl, alkoxy and aryloxy group, which may be linear, branched, cyclic, polycyclic, unsaturated, hydroxylated, carbonylated, phosphorylated and/or sulfurized, said group possibly possessing in its backbone a heteroatom particularly O, S and/or or N, and Pro* corresponding to Proline, an analogue or derivative thereof; comprising, for example, Myr-PPL-OH and Myr-PPR-OH.

Here can further be cited also the propigmenting and/or pro-mec dipeptides and tripeptides of general Formula X-(Xaa₁)ₙ-Pro*-Xaa₂-Y disclosed in WO2014/080376, with n=0, 1 or 2, Xaa₁ an hydrophobic aminoacid selected from Ala, Val, Met, Leu, Iso, Phe, Pro, and analogs and derivatives thereof; or a polar aminoacid selected from Ser, Thr, Tyr, Asp, Glu and analogs and derivatives thereof; and when n=2 the two aminoacids Xaa₁ being the same or different; Xaa₂ being an hydrophobic aminoacid selected from Ala, Val, Met, Leu, Iso, Phe, and analogs and derivatives thereof, or a basic aminoacid selected from Arg, Lys, His, and analogs and derivatives thereof; at the N terminal end X being selected from H, —CO—R₁ and —SO₂—R₁; at the C terminal end Y being selected from OH, OR₁, NH₂, NHR₁ or NR₁R₂; R₁ and R₂ being, independently from each other, selected from an alkyl, aryl, aralkyl, alkylaryl, alkoxy et aryloxy group, that can be linear, branched, cyclic polycyclic, saturated, unsaturated, hydroxylated, carbonylated, phosphorylated and/or sulfured, said group having or not an O, S and/or N heteroatom in its skeleton and Pro* corresponding to a Proline, analog or derivative thereof; comprising for example the following peptides Pal-SPR-OH, Pal-PPR-OH, Pal-QPA-OH, Pal-LPAOH, Myr-SPA-OH, Pal-PM-OH, Pal-PA-OH and Pal-PP-OH.

Suitable tetrapeptide derivatives for use as additional peptides according to the present invention include, but are not limited to, Pal-GQPR (SEQ ID NO: 2) (from Sederma), Pal-KTFK (SEQ ID NO: 6) or Ela-KTFK (SEQ ID NO: 18), Ela-KTAK (SEQ ID NO: 19), Ela-KAYK (SEQ ID NO: 20) or Ela-KFYK (SEQ ID NO: 21). Suitable pentapeptide derivatives for use as additional peptides herein include, but are not limited to Pal-KTSKS (SEQ ID NO: 7), Pal-YGGFXaa (SEQ ID NO: 22) with Xaa being Leu or Pro, or mixtures thereof.

Suitable hexapeptide derivatives for use herein include, but are not limited to, Pal-VGVAPG (SEQ ID NO: 3), Pal-GKTTKS (SEQ ID NO: 6), Pal-GKTSKS (SEQ ID NO: 23), Pal-HLDIIXaa with Xaa being Trp, Phe, Tyr, Tic, 7-hydroxy-Tic ou Tpi (SEQ ID NO: 24) and derivatives thereof. The mixture of Pal-GHK and Pal-GQPR (SEQ ID NO: 2) (Matrixyl® 3000, Sederma) can also be mentioned.

The preferred compositions commercially available containing a tripeptide or a derivative include Biopeptide-CL™, Maxilip™, Biobustyl™, Procapil™ and Matrixyl® synthe'6® of Sederma. The compositions commercially available preferred sources of tetrapeptides include Rigin™, Eyeliss™ Matrixyl® Reloaded and Matrixyl 3000® which contain between 50 and 500 ppm of Pal-GQPR (SEQ ID NO: 2), Crystalide™ and an excipient, proposed by Sederma.

The following marketed peptides can be mentioned as well as additional active ingredients:
Vialox™ (INCI name=Pentapeptide-3 (synthetic peptide comprising alanine, arginine, isoleucine, glycine and proline)), Syn-ake™ (β-Ala-Pro-Dab-NH-Bzl) or Syn-Coll™ (Pal-Lys-Val-Lys-OH) sold by Pentapharm;
Argireline™ (Ac-Glu-Glu-Met-Gln-Arg-Arg-NH₂ (INCI name=Acetyl hexapeptide-3) (SEQ ID NO: 25), Leuphasyl™ (Tyr-D-Ala-Gly-Phe-Leu) (SEQ ID NO: 26), Aldenine™ (Gly-His-Lys), Trylagen™ (INCI name=Pseudoalteromonas Ferment Extract, Hydrolyzed Wheat Protein, Hydrolyzed Soy Protein, Tripeptide-10 Citrulline (reaction product of Citrulline and Tripeptide-10 (synthetic peptide constituted of aspartic acid, isoleucine and lysine)), Tripeptide-1), Eyeseryl™ (Ac-β-Ala-His-Ser-His)(SEQ ID NO: 27), Serilesine™ (Ser-Ile-Lys-Val-Ala-Val) (SEQ ID NO 28) or Decorinyl™ (INCI name: Tripeptide-10 Citrulline=reaction product of Citrulline and Tripeptide-10 (synthetic peptide constituted of aspartic acid, isoleucine and lysine) sold by Lipotec;
Collaxyl™ (Gly-Pro-Gln-Gly-Pro-Gln (SEQ ID NO 29)) or Quintescine™ (Cys-Gly) sold by Vincience;
Cytokinol™ LS (casein hydrolysate) sold by Les Laboratoires Serobiologiques/Cognis;
Kollaren™ (Gly-His-Lys), IP2000™ (Pal-Val-Tyr-Val) or Meliprene™ (INCI name=Monofluoroheptapeptide-1: reaction product of acetic acid and a synthetic peptide comprising arginine, glycine, glutamic acid, histidine, norleucine, p-fluorophenylalanine and tryptophan) sold by l'lnstitut Européen de Biologie Cellulaire;
Neutrazen™ (Pal-His-D-Phe-Arg-NH₂) sold by Innovations; or
BONT-L-Peptide™ (INCI name=Palmitoyl Hexapeptide-19: reaction product of palmitic acid and Hexapeptide-19 (synthetic peptide constituted of asparagine, aspartic acid, lysine and methionine), Timp-Peptide™ (INCI name=Acetyl Hexapeptide-20: reaction product obtained by acetylation of Hexapeptide-20 (synthetic peptide constituted of alanine, glycine, lysine, valine and proline) or ECM Moduline™ (INCI name=Palmitoyl Tripeptide-28: reaction product of palmitic acid and Tripeptide-28 (synthetic peptide constituted of arginine, lysine and phenylalanine) sold by lnfinitec Activos.

It is also possible to envisage combining the plant cells according to the invention with one or more cyclic peptides, in particular those extracted from linseed oil described in the Applicant's patent application FR1850845.

Different compositions/formulations according to the invention are described below with some examples of additional active ingredients.

The active ingredient according to the invention is as described in point C/ above comprising 100 ppm of peptide(s) according to the invention.

This ingredient is generally formulated within a range of 1 to 5%, preferably 3%.
1) Cream Form, for Example an Anti-Aging Day Cream for the Face

TABLE 20

| Raw materials | INCI name | Function | % |
|---|---|---|---|
| Part A: | | | |
| H₂O | / | / | qsp100 |
| Carbopol ™ Ultrez 10 | Carbomer | Thickener/ | 0.30 |
| | | gelling agent | |
| Part B: | | | |
| Brij S2-SS-(RB) ™ | Steareth-2 | Emulsifier | 0.40 |
| Brij S10-SO-(RB) ™ | Steareth-10 | Emulsifier | 1.20 |
| Crodafos | Cetearyl Alcohol (and) | Emulsifier/ | 4.00 |
| CES-PA-(RB) ™ | Dicetyl Phosphate (and) | conditionner | |
| | Ceteth-10 Phosphate | | |
| Crodacol | Cetearyl Alcohol | Emollient | 150 |
| CS90-PA-(RB) | | | |
| Laurocapram | Laurocapram | Emollient | 2.50 |
| Crodamol ™ | C12-15 Alkyl Benzoate | Emollient | 1.50 |
| AB-LQ-(RB) | | | |
| Crodamol ™ | Diethylhexyl Succinate | Emollient | 7.00 |
| OSU-LQ-(JP) | | | |
| Part C: | | | |
| Glycerin | Glycerin | Humectant | 2.50 |
| Octanediol | Caprylyl Glycol | Humectant/ | 0.50 |
| | | Emollient | |
| Part D: | | | |
| Phenoxyethanol | Phenoxyethanol | Preservative | qs |
| Part E: | | | |
| Potassium sorbate | Potassium Sorbate | Preservative | qs |
| Part F: | | | |
| H₂O | / | / | 4.00 |
| NaOH 30% | Sodium Hydroxide | pH adjuster | 0.40 |
| Part G: | | | |
| Ingredient according | / | Active | 3.00 |
| to the invention | | | |

### Example(s) of Additional Ingredient(s)

1. a moisturizing/smoothing ingredient such as:
   OPTIM HYAL™, marketed by Sederma, contains oligosaccharides of acetylated glucuronic acids having a structure analogous to fragments of hyaluronic acid.
2. a sebum-regulating ingredient such as:
   SEBULESS™, marketed by Sederma, comprising an extract of Syringa vulgaris obtained by in vitro cell culture, purifying sebum regulator, mattifies and refreshes the complexion, blurs imperfections.
   PORETECT™, marketed by Sederma, comprising a combination of linseed and celery extracts titrated in cylolinopeptides and senkyunolides, which brings firmness, tone and density to the skin, thus strengthening the pore-supporting structures sagging with age.
3. In activity reinforcement: an ingredient acting on the elastic properties of the skin/skin barrier such as:
   IDEALIFT™, marketed by Sederma, comprising the lipodipeptide N-acetyl-Tyrosyl-Arginyl-O-hexadecyl ester, combating facial flaccidity and improving resistance to gravity, in particular via the stimulation of elastin.
   DERMAXYL™, marketed by Sederma, combining ceramide 2, cement of the stratum corneum, and a palmitoylated matrikine Pal-Val-Gly-Val-Ala-Pro-Gly, which smoothes wrinkles and repairs the skin barrier.

2) Mild Aqueous Serum Form

TABLE 21

| Raw materials | INCI name | Function | % |
|---|---|---|---|
| Part A: | | | |
| H₂O | / | / | qsp100 |
| Potassium sorbate | Potassium Sorbate | Preservative | 0.10 |
| Part B: | | | |
| Glycerin | Glycerin | Humectant | 5.00 |
| Phenoxyethanol | Phenoxyethanol | Preservative | 0.80 |
| Part C: | | | |
| Cromollient ™ | Di-PPG-2 Myreth-10 | Emollient | 1.20 |
| SCE | Adipate | | |
| VisiaOptima ™ | Sodium Polyacrylate (and) | Rheology | 1.00 |
| SE | Ethylhexyl Cocoate (and) | modifier | |
| | PPG-3 Benzyl Ether | and emulsion | |
| | Myrisate (and) | stabilizer | |
| | Polysorbate 20 | | |
| Part D: | | | |
| H₂O | / | / | 0.80 |
| NaOH 30% | Sodium Hydroxide | pH adjuster | 0.08 |
| Part C: | | | |
| Ingredient according | / | Active | 3.00 |
| to the invention | | | |

### Example(s) of Additional Ingredient(s)

4. An anti-aging ingredient such as:
   SENESTEM™, marketed by Sederma, comprising plant cells obtained by in-vitro cell culture of Plantago lanceolata, which in particular improves the viscoelastic properties of the skin and lightens the pigmentary spots of senescence.
5. An antioxidant agent such as:
   MAJESTEM™, marketed by Sederma, based on Leontopodium alpinum plant cells obtained by in-vitro cell culture titrated in leontopodic acid; neutralizes oxidative stress (pollution, UV radiation) and restores skin tension.

3) Gel Form

TABLE 22

| Raw materials | INCI name | Function | % |
|---|---|---|---|
| Part A: | | | |
| H₂O | / | / | qsp100 |
| Carbomer | / | Rheology | 0.40 |
| | | modifier | |
| Part B: | | | |
| Glycerin | Glycerin | Humectant | 7.00 |
| Phenoxyethanol | Phenoxyethanol | Preservative | 0.80 |
| Part C: | | | |
| H₂O | / | / | 3.00 |
| NaOH 30% | Sodium Hydroxide | pH adjuster | 0.30 |
| Part D: | | | |
| Tween ™ 20 | Polysorbate 20 | Emulsifier | 0.50 |
| Cromollient ™ | Di-PPG-2 Myreth-10 | Emollient | 1.00 |
| SCE | Adipate | | |
| Covi-ox ™ | Tocopherol (and) | Antioxidant | 0.40 |
| | Helianthus Annuus | | |
| | (Sunflower) Seed Oil | | |
| Part E: | | | |
| Ingredient according | / | Active | 3.00 |
| to the invention | | | |

### Example(s) of Additional Ingredient(s)

6. an “antipollution” ingredient such as:
   CITYSTEM™, marketed by Sederma, based on plant cells obtained in vitro of Marrubium vulgare with a high concentration of Forsythoside B; used against pollution attacks: makes the skin soft and smooth, refines the skin texture, reduces the visibility of comedones, leaving the skin radiant and purified.
7. a calming ingredient for sensitive skin such as:
   PACIFEEL™, marketed by Sederma, comprising an extract of Mirabilis Jalapa.
8. An hydrating ingredient such as:
AQUALANCE™, marketed by Sederma, osmoprotective hydrating active ingredient composed of homarin and erythritol.

4) Gel Form to Realise a Spray Mask

TABLE 23

| Raw materials | INCI name | Function | % |
|---|---|---|---|
| Part A: | | | |
| H₂O | / | / | qsp100 |
| Hydrotriticum | Aqua (and) Hydrolyzed | Film forming | 3.00 |
| PVP PE ™ | Wheat Protein/PCP | agent | |
| | Crosspolymer | | |
| Volarest ™ FL | Acrylates/Beheneth-25 | Rheology | 2.30 |
| | Methacrylate Copolymer | modifier | |
| Potassium sorbate | Potassium Sorbate | Preservative | |
| Part B: | | | |
| Glycerin | Glycerin | Humectant | 5.00 |
| Phenoxyethanol | Phenoxyethanol | Preservative | 0.80 |
| Part C: | | | |
| Crovol ™ A70 | PEG-60 Almond Glycerides | Emollient | 1.00 |
| Ethanol | Ethanol | Solvent | 5.00 |
| Covi-ox ™ | Tocopherol (and) | Antioxidant | 0.20 |
| | Helianthus Annuus | | |
| | (Sunflower) Seed Oil | | |
| Part D: | | | |
| H₂O | / | / | 2.50 |
| NaOH 30% | Sodium Hydroxide | pH adjuster | 0.25 |
| Part E: | | | |
| Ingredient according | | Active | 3.00 |
| to the invention | | | |

### Example(s) of Additional Ingredient(s)

9. an ingredient that acts on the radiance of the complexion such as:
   EVERMAT™, marketed by Sederma, comprising a combination of an extract of Enantia chlorantha rich in protobberins and oleanolic acid; decreases pore size and shining; refines the texture of acne-prone skin.
10. an ingredient with revitalizing properties such as:
   Fruitliquid™ Kumquat™, marketed by Crodarom.

5) Cream Form, for a Make-Up Base

TABLE 24

| Raw materials | INCI name | Function | % |
|---|---|---|---|
| Part A: | | | |
| H₂O | / | / | qsp 100 |
| Volarest ™ FL | Acrylates/Beheneth-25 | Rheology | 0.90 |
| | Methacrylate | modifier | |
| | Copolymer | | |
| Part B: | | | |
| Arlacel ™ 2121 | Sorbitan Stearate (and) | Emulsifier | 4.50 |
| | Sucrose Cocoate) | | |
| Part C: | | | |
| Pentylene glycol | Pentylene Glycol | Humectant | 5.00 |
| Phenoxyethanol | Phenoxyethanol | Preservative | 0.80 |
| Part D: | | | |
| Crodamol ™ SSA | Decyl Isostearate (and) | Emollient | 2.00 |
| | Isostearyl Isostearate | | |
| Crodamol ™ TN | Isotridecyl Isononanoate | Emollient | 2.00 |
| Crodamol ™ AB | C12-C15 Alkyl Benzoate | Emollient | 1.50 |
| Crodamol ™ GTEH | Triethylhexanoin | Emollient | 3.00 |
| Covi-ox ™ | | Antioxidant | 0.10 |
| | Tocopherol (and) | | |
| | Helianthus Annuus | | |
| | (Sunflower) Seed Oil | | |
| Part D: | | | |
| Potassium sorbate | Potassium Sorbate | Preservative | 0.10 |
| Part E: | | | |
| H₂O | / | / | 2.50 |
| NaOH 30% | Sodium Hydroxide | pH adjuster | 0.25 |
| Part E: | | | |
| Ingredient according | | Active | 3.00 |
| to the invention | | | |

### Example(s) of Additional Ingredient(s)

11. A concealer/eye contours ingredient such as:
   HALOXYL™, marketed by Sederma, combination of 2 matrikines, Pal-GHK and Pal-GQPR with N-hydroxysuccinimide and a flavonoid, the chrysin.
   EYELISS™, marketed by Sederma, combines three components: hesperidin methyl chalcone, the dipeptide Valyl-Tryptophan (VW) and the lipopeptide Pal-GQPR.
   PRODIZIA™, marketed by Sederma, comprising an extract of Albizia julibrissin, which promotes the visible reduction of signs of fatigue: dark circles, under eyes bags, dull complexion and drawn lines by repairing and protecting the skin from damage caused by glycation.
12. An anti-wrinkle/anti-aging ingredient based on peptide(s) such as: MATRIXYL 3000™
   MATRIXYL synthe'6™ and/or MATRIXYL Morphomics™, marketed by Sederma.

1. Use of at least one peptide of the following general Formula 1:
X-(Xaa)ₙK*TTK*X′aa-(Xaa)ₘ-Z
for a non-therapeutic cosmetic treatment of keratinous tissues of the skin and of its appendages, wherein in the general Formula 1:
K* is selected from lysine, hydroxylysine, ornithine, diaminobutyric acid or diaminopropionic acid or their formylated, acetylated, trifluoroacetylated, methanesulfonylaed or succinylated derivatives, both K* being identical or different;
(Xaa)n and (Xaa)m corresponding independently of one another to a sequence of n or m amino acids Xaa selected independently of one another from Gly, Ala, Pro, Val, Leu, Ile and Phe, with n and m being integers which can be equal or different and comprised between 0 and 5;
X′aa is selected from threonine and serine;
At the N-terminal end X is selected from H, —CO—R¹, —SO₂—R¹ or a biotinoyle group;
At the C-terminal end Z is selected from OH, OR¹, NH₂, NHR¹ or NR¹R²; and
R¹ and R² being, independently of one another, selected from an alkyl, aryl, aralkyl, alkylaryl, alkoxy, saccharide and aryloxy group, which can be linear, branched, cyclic, polycyclic, unsaturated, hydroxylated, carbonylated, phosphorylated and/or sulfurated, said group having from 1 to 24 carbon atoms and the possibility of having in its backbone one or more O, S and/or N heteroatoms.

2. The use according to claim 1, wherein the treatment is adapted to preserve, protect and/or improve the condition of the epidermis by strengthening the skin barrier function and harmonizing the natural process of epidermal maturation.

3. The use according to claim 1, wherein the treatment is adapted to protect the skin and its appendages from molecules and aggressions of the external environment including the defense against bacteria, inflammation, and radiations.

4. The use according to claim 1, wherein the treatment is adapted to improve the hydration of the upper part of the epidermis by reducing the transepidermal water loss (TEWL).

5. The use according to claim 1, wherein the treatment is adapted to treat the epidermal scars.

6. The use according to claim 1, wherein the treatment is adapted to treat epidermal acne scars.

7. The use according to claim 1, wherein the treatment is adapted to beautify nails, hair and body hair including eyelashes and eyebrows.

8. The use according to claim 3, wherein the treatment is adapted to fight against the microorganisms of acne and/or of dandruff state.

9. The use according to claim 1, wherein the at least one peptide is used in a vectorized form, being bound, incorporated or adsorbed on/to macro-, micro-, or nano-particles in the form micro- or nano-emulsions, or adsorbed on powdery organic polymers, talcs, bentonites, spores or exins and other inorganic or organic supports.

10. The use of at least one peptide having the following general Formula 1:
X-(Xaa)nK*TTK*X′aa-(Xaa)m-Z
wherein in the general Formula 1:
K* is selected from lysine, hydroxylysine, ornithine, diaminobutyric acid or diaminopropionic acid or their formylated, acetylated, trifluoroacetylated, methanesulfonylaed or succinylated derivatives, both K* being identical or different;
(Xaa)n and (Xaa)m corresponding independently of one another to a sequence of n or m amino acids Xaa selected independently of one another from Gly, Ala, Pro, Val, Leu, Ile and Phe, with n and m being integers which can be equal or different and comprised between 0 and 5;
X′aa is selected from threonine and serine;
At the N-terminal end X is selected from H, —CO—R¹, —SO₂—R¹ or a biotinoyle group;
At the C-terminal end Z is selected from OH, OR¹, NH₂, NHR¹ or NR¹R²; and
R¹ and R² being, independently of one another, selected from an alkyl, aryl, aralkyl, alkylaryl, alkoxy, saccharide and aryloxy group, which can be linear, branched, cyclic, polycyclic, unsaturated, hydroxylated, carbonylated, phosphorylated and/or sulfurated, said group having from 1 to 24 carbon atoms and the possibility of having in its backbone one or more 0, S and/or N heteroatoms.
for the preparation of a composition for treatment of keratinous tissues of the skin and of its appendages.

11. The use according to claim 1, wherein K* is lysine or ornithine.

12. The use according to claim 1, wherein X′aa is serine.

13. The use according to claim 1, wherein n and m are independently of one another 0 or 1 or 2.

14. The use according to claim 1, wherein the peptide is either modified at the N-terminal position or at the C-terminal position.

15. The use according to claim 1, wherein R¹ and/or R² is an alkyl chain of 1 to 24 carbon atoms.

16. The use according to claim 1, wherein R¹ and/or R² is an alkyl chain of 3 to 24 carbon atoms.

17. The use according to claim 1, wherein X is a CO—R¹ acyl group and Z is selected from OH, OMe, OEt and NH₂.

18. The use according to claim 1, wherein the peptide is the Pal-KTTKS (SEQ ID NO 1).

19. Method of skin treatment comprising topical application to a skin in need thereof of an effective amount of the peptide as defined in claim 1 wherein the treatment is antimicrobial (antibacterial or antifungal), and/or anti-inflammatory, said treatment being suitable for soothing sensitive and irritated skin and/or for treating acne, psoriasis, dermatitis and eczema.


SEQUENCE LISTING (28 sequences)

SEQ ID NO: 1 - protein, 5 aa, artificial sequence
  synthetic peptide
  MOD_RES (1)..(1): amidation by a Palmitoyl chain on the N terminal end
KTTKS

SEQ ID NO: 2 - protein, 4 aa, artificial sequence
  synthetic peptide
  MOD_RES (1)..(1): amidation by a Palmitoyl chain on the N terminal end
GQPR

SEQ ID NO: 3 - protein, 6 aa, artificial sequence
  synthetic peptide
  MOD_RES (1)..(1): amidation by a Palmitoyl chain on the N terminal end
VGVAPG

SEQ ID NO: 4 - protein, 5 aa, artificial sequence
  peptidic compound
  MISC_FEATURE (1)..(1): Xaa being a lysine, hydroxylysine, ornithine, ldiaminobutyric acid,diaminopropionic acid or formyl, acetyl, trifluoroacetyl, methanesulfonyle or succinyl derrivatives
  MOD_RES (1)..(1): X = -CO-R1 or -SO2-R1 or biot group, R1 = C1-C24 group
  MISC_FEATURE (4)..(4): Xaa being a lysine, hydroxylysine, ornithine, ldiaminobutyric acid,diaminopropionic acid or formyl, acetyl, trifluoroacetyl, methanesulfonyle or succinyl derrivatives
  MISC_FEATURE (5)..(5): Xaa being a serine or a threonin
  MOD_RES (5)..(5): Z = -OR1, -NH2, -NHR1 ou -NR1R2 ; R1, R2 = C1-C24 group
XTTXX

SEQ ID NO: 5 - protein, 6 aa, artificial sequence
  synthetic peptide
  MOD_RES (1)..(1): amidation by a Palmitoyl chain on the N terminal end
GKTTKS

SEQ ID NO: 6 - protein, 4 aa, Artificial sequence
  Synthetic peptide
  MOD_RES (1)..(1): amidation by a Palmitoyl chain on the N terminal end
KTFK

SEQ ID NO: 7 - protein, 5 aa, artificial sequence
  synthetic peptide
  MOD_RES (1)..(1): amidation by a Palmitoyl chain on the N terminal end
KTSKS

SEQ ID NO: 8 - protein, 4 aa, artificial sequence
  synthetic peptide
RSRK

SEQ ID NO: 9 - protein, 4 aa, artificial sequence
  synthetic peptide
GQPR

SEQ ID NO: 10 - protein, 4 aa, artificial sequence
  peptidic compound
KTFK

SEQ ID NO: 11 - protein, 4 aa, Artificial sequence
  Synthetic peptide
KTAK

SEQ ID NO: 12 - protein, 4 aa, Artificial sequence
  Synthetic peptide
KAYK

SEQ ID NO: 13 - protein, 4 aa, Artificial sequence
  Synthetic peptide
KFYK

SEQ ID NO: 14 - protein, 5 aa, artificial sequence
  synthetic peptide
KTSKS

SEQ ID NO: 15 - protein, 6 aa, artificial sequence
  synthetic peptide
GKTTKS

SEQ ID NO: 16 - protein, 6 aa, artificial sequence
  synthetic peptide
GKTSKS

SEQ ID NO: 17 - protein, 6 aa, artificial sequence
  synthetic peptide
VGVAPG

SEQ ID NO: 18 - protein, 4 aa, artificial sequence
  synthetic peptide
  MOD_RES (1)..(1): amidation by a Elaidoyl chain on the N terminal end
KTFK

SEQ ID NO: 19 - protein, 4 aa, Artificial sequence
  Synthetic peptide
  MOD_RES (1)..(1): amidation by a Elaidoyl chain on the N terminal end
KTAK

SEQ ID NO: 20 - protein, 4 aa, Artificial sequence
  Synthetic peptide
  MOD_RES (1)..(1): amidation by a Elaidoyl chain on the N terminal end
KAYK

SEQ ID NO: 21 - protein, 4 aa, Artificial sequence
  Synthetic peptide
  MOD_RES (1)..(1): amidation by a Elaidoyl chain on the N terminal end
KFYK

SEQ ID NO: 22 - protein, 5 aa, artificial sequence
  synthetic peptide
  MOD_RES (1)..(1): amidation by a Palmitoyl chain on the N terminal end
  MISC_FEATURE (5)..(5): Xaa being either a Proline P or a Leucine L.
YGGFX

SEQ ID NO: 23 - protein, 6 aa, Artificial sequence
  Synthetic peptide
  MOD_RES (1)..(1): amidation by a Palmitoyl chain on the N terminal end
  MISC_FEATURE (6)..(6): Xaa being Trp, Phe, Tyr, Tic, 7-hydroxy-Tic or Tpi
HLDIIX

SEQ ID NO: 24 - protein, 6 aa, artificial sequence
  synthetic peptide
  MOD_RES (1)..(1): Acetylation on the N-terminal end
EEMQRR

SEQ ID NO: 25 - protein, 5 aa, artificial sequence
  synthetic peptide
YAGFL

SEQ ID NO: 26 - protein, 4 aa, artificial sequence
  synthetic peptide
AHSH

SEQ ID NO: 27 - protein, 6 aa, artificial sequence
  synthetic peptide
SIKVAV

SEQ ID NO: 28 - protein, 6 aa, artificial sequence
  synthetic peptide
GPQGPQ